(19) United States
(12) Patent Application Publication
(10) Pub. No.: US 2021/0065893 A1
(43) Pub. Date: Mar. 4, 2021

(54) Method to Encourage Patient Feedback on Interaction with Device

(71) Applicant: Biotronik SE & Co. KG, Berlin (DE)

(72) Inventors: Larry STOTTS, Tigard, OR (US); Dawn Gayle FLAKNE, Portland, OR (US); Dirk MUESSIG, West Linn, OR (US)

(21) Appl. No.: 16/961,783

(22) PCT Filed: Dec. 12, 2018
(86) PCT No.: PCT/EP2018/084538

§ 371 Date: Jul. 13, 2020

Related U.S. Application Data
(60) Provisional application No. 62/621,061, filed on Jan. 24, 2018.

(51) Int. Cl. G16H 40/67; G16H 10/60; G16H 40/20; A61B 5/042; A61B 5/00; A61B 5/021; H04L 29/08; G08C 17/02

(52) U.S. Cl. CPC G16H 40/67; G16H 10/60; G16H 40/20; A61B 5/042; G08C 17/02; A61B 5/686; A61B 5/02141; H04L 67/12; A61B 5/0006

(57) ABSTRACT

A system for collecting information concerning a patient's health status at an event, comprising: a medical device recording physiological data of a patient, a remote control device allowing the patient to initiate said recording of physiological data at an event, wherein the remote control device sends a control signal to the medical device in response to a corresponding operation of the remote control device by the patient for initiating a recording of physiological data by the medical device, wherein the medical device records said physiological data when receiving said control signal, and a patient device that prompts the patient after initiation of said recording to input information on the patient's health status at the time of said event that lead the patient to initiate recording of physiological data. Further, the present invention relates to a method for collecting information concerning a patient's health status.

The invention relates to a system and a method for collecting information concerning a patient's health status at an event. Such an event can be any occasion at which the patient/user of the system decides to initiate a recording of physiological data (e.g. an ECG) by means of a medical device (e.g. an implantable cardiac pacemaker or a loop recorder etc.).

Currently, patients can cause an implant to record certain information when they are feeling unwell. Particularly, U.S. Pat. No. 8,600,506 B2 describes a method and a system for collecting patient event information, where the system includes an implantable medical device (IMD) and an external interface device.

Based on the above it is an objective of the present invention to provide an improved system and method for collecting information concerning a patient's health status.

Particularly, it is an objective of the present invention to provide a mechanism for the patient to generate a record of time, state and perceived feeling when triggering a recording of physiological data due to a perceived event. Furthermore, it is desired that the mechanism also allows the patient to get real time information of the e.g. cardiac condition when he/she is experiencing an event that led him/her to trigger a recording by the implanted medical device.

According to claim 1, a system for collecting information concerning a patient's health status at an event is disclosed, the system comprising:
medical device configured for recording of physiological data of a patient,
a remote control device for allowing the patient to initiate said recording of physiological data at an event, wherein the remote control device is configured to send a control signal to the medical device in response to a corresponding operation of the remote control device by the patient for initiating a recording of physiological data by the medical device, wherein the medical device is configured to record said physiological data when receiving said control signal, and
a patient device (e.g. for receiving the recorded physiological data), wherein the patient device is configured to prompt the patient after initiation of said recording to input information on the patient's health status (herein also denoted as health survey data) at the time of said event that lead the patient to initiate said recording of physiological data.

According to an embodiment of the present invention, the medical device is an implantable medical device.

Particularly, the remote control device can be a special purpose device. However, the remote control device can also be formed by some other (e.g. portable) device, such as a smart phone, a tablet computer etc. Furthermore, the patient device can be some device accessible by the patient such as a computer (e.g. a tablet computer, a desktop computer, a laptop etc.), a smart phone, or another suitable device.

Particularly, the present invention provides a way to invoke a reminder and a mechanism for the patient to record their state of health in an external device when the implant is instructed to record its information. Particularly, in addition, this invention can cause the implant to transmit its recorded data to an external service allowing the patient to see the information immediately.

Furthermore, particularly, the same patient action that causes the medical device to start a recording of physiological data (e.g. ECG) also triggers the external patient device to open a reminder to record non implantable device related health information. Particularly, when the patient acknowledges the reminder, the external patient device automatically brings up a form for health information entry. The patient may enter information by answering questions, in free form text and/or vocalization and/or photos and/or video. When completed, the information may be stored on the external patient device for in physician office review, sent directly to the clinician using various different mechanisms (e.g., email, fax, SMS, etc.) and/or sent to an alternate data storage facility/mechanism (e.g., a secure cloud or data service center) for later review. In addition, the patient entered record can be marked in a way that can be correlated with the recording by the medical device (e.g. the message may be timestamped and this timestamp can be correlated with the timestamp of the recording of the medical device).

Furthermore, the external device may receive information from the medical device about the specific of the patient (e.g. heart rhythm abnormalities like atrial fibrillation), pass the information on to the treating physician, or external storage, or displays the information (or a subset) directly to the patient. Such information may furthermore trigger a (self-)treatment of the patient (e.g. taking of medication or modifying medication).

Particularly, according to a further embodiment of the system according to the present invention, said recorded physiological data is an ECG of the patient (e.g. measured by the medical device) or a pressure signal (e.g. measured via pressure sensor in the right ventricular outflow tract RVOT).

Further, according to an embodiment of the system according to the present invention, the patient device is configured to record said information on the patient's health status in form of a text and/or as an audio signal and/or as photos and/or as video. Particularly, the patient may enter information by answering questions, in free form text or vocalization, see also above.

Further, according to an embodiment of the system according to the present invention, the remote control device and patient device may be formed by a single device or may be designed as separate devices.

Furthermore, according to an embodiment of the system according to the present invention, the patient device is configured to at least one of: store recorded physiological data, store said information on the patient's health status provided by patient, send recorded physiological data and/or said information on patient's health status provided by the patient to a clinician and/or a data storage facility (e.g., a secure cloud or data service center).

Further, according to an embodiment of the system according to the present invention, for associating the recorded physiological data with said information on the patient's health status, the patient device is configured to provide said information on the patient's health status with an identifier (e.g., a timestamp), and the medical implant is configured to provide the recorded data with a correlated identifier, too.

Further, according to an embodiment of the system according to the present invention, the remote control device comprises a control element (e.g. a button or some other form of user interface), and a means of wireless communication, e.g. a transmitter, wherein the remote control device is configured to send said control signal via a wireless link using said transmitter when the patient actuates said control element to the medical device, indicating that the medical device should activate a recording of physiological data of the patient.

Further, according to a further embodiment of the system according to the present invention, the remote control device is configured to send a message via a wireless link (e.g. via Bluetooth, Bluetooth LE, NFC, etc.) to the patient device (e.g. a portable device such as a smartphone etc.) in order to notify it that a recording of physiological data of the patient has been initiated, wherein the patient device is configured to display a notification to the patient and to prompt the patient to acknowledge said notification, and wherein when the patient acknowledges said notification, the patient device prompts the patient to input said information on the patient's health status, e.g. brings up a patient health survey that allows the patient to enter their status when the event happened.

Further, particularly, the patient may enter the information in a variety of ways (e.g., button pushes, text, speech, photos, video or a combination). When done, the patient may send the health survey data to the clinician via a variety of mechanisms (e.g., e-mail, fax, etc.)

Further, particularly, the patient device may also send the information automatically to a data storage facility (such as a secure cloud or data service center). Alternatively or in addition, the health survey data may be stored on the patient device for later review at the clinician's office. The medical device may keep a record of the recorded event for later review by the clinician and/or may also send its recorded information to the data storage facility. The information from the medical device and the external patient device may be correlated and presented to the clinician as a cohesive data set.

Further, according to an embodiment of the system according to the present invention, the medical device is configured to send the recorded physiological data to the remote control device a via wireless link, wherein the remote control device is configured to send the recorded physiological data to the patient device via a wireless link, too.

The patient device may extract some or all information from the medical device to display it to the patient, and send the data from the medical device combined/in addition with the health survey data (i.e. said information on the patient's health status at the time of said event) to the clinician and or to said data storage facility (e.g. cloud or data service center).

Furthermore, according to an embodiment of the system according to the present invention, the remote control device is configured to initiate said recording by sending said control signal via a wireless link to the medical device, wherein the remote control device is further configured to trigger the patient device to start listening for a data transmission from the medical device by sending a further control signal via a wireless link to the patient device, wherein the medical device is configured to wirelessly transmit said recorded physiological data to the patient device.

Further, particularly, the patient device may receive the data from the medical device, trigger the patient to provide said health survey information (i.e. said information on the patient's health status at the time of said event) and sends all information to the clinician and/or to a data storage facility (e.g. a secure cloud or data service center). In addition, the patient device may extract some or all information from the medical device to display it to the patient.

According to yet another aspect of the present invention a method for recording physiological data and information on a patient's health status (e.g. at the time of initiation of said recording) is disclosed. Particularly, the method may use a system according to the present invention. The method according to the present invention comprises the steps of:
initiating recording of physiological data of a patient by an medical device, wherein said recording is initiated by sending a control signal by a remote control device to the medical device,
after initiation of said recording prompting the patient to input information on the patient's health status at the time of an event that lead the patient to initiate said recording of physiological data.

Particularly, the recorded physiological data may be sent to a patient device and/or to a data storage facility. Further, the medical device may store said recorded data (for later review).

Particularly, according to an embodiment of the method according to the present invention, said physiological data is an electrocardiogram (ECG).

Further, according to an embodiment of the method according to the present invention, said information on the patient's health status is input into the patient device in form of text, audio signal, photos and/or video (see also above).

Further, according to an embodiment of the method according to the present invention, the method further comprise at least one of the following steps: storing recorded physiological data in the patient device, storing said information on the patient's health status provided by patient in the patient device, prompting the patient device to send recorded physiological data and/or said information on the patient's health status provided by the patient to a further data storage facility (e.g., a secure cloud or data service center).

Further, according to an embodiment of the method (and system) according to the present invention, said information on the patient's health status is automatically provided with an identifier (e.g., a timestamp and/or GPS location, etc.), and the recorded physiological data is automatically provided with a correlated identifier, too. Particularly the respective identifier is indicative of the respective event that led to a recording of physiological data of the patient. Further, particularly, the respective indicator may also be indicative of the location at which said event took place.

Further, according to an embodiment of the method according to the present invention, said control signal is sent in a wireless fashion to the medical device, indicating that the medical device should activate a recording of physiological data of the patient.

Further, according to an embodiment of the method according to the present invention, the method comprises the further steps of: sending a message via a wireless link (e.g. via Bluetooth, Bluetooth LE, NFC, etc.) from the remote control device to the patient device (e.g. a portable device such as a smartphone etc.) in order to notify the patient device that a recording of physiological data of the patient has been initiated, displaying a notification to the patient using the patient device and prompting the patient to acknowledge said notification, and wherein when the patient acknowledges said notification, prompting the patient to input said information on the patient's health status.

Further, according to an embodiment of the method according to the present invention, the method comprises the further steps of: sending the recorded physiological data from the medical device to the remote control device via a wireless link, and sending the recorded physiological data from the remote control device to the patient device via a wireless link.

Furthermore, according to an embodiment of the method according to the present invention, the method further comprises the steps of: initiating said recording by sending said control signal via a wireless link from the remote control device to the medical device, triggering the patient device to start listening for a transmission of said recorded physiological data from the medical device by sending a further control signal via a wireless link to the patient device, and transmitting said recorded physiological data in a wireless fashion from the medical device to the patient device.

The present invention allows the user/patient to be reminded to record their state of health coincident to an event that caused the patient to request the medical device to record its data. Furthermore, the recorded data can be sent to the clinician and/or to the service center and correlated with the data from the medical device to give a more complete picture to the clinician. In addition, the medical device's data may also be presented to the patient.

Further features and embodiments of the present invention shall be described below with reference to the Figures, wherein

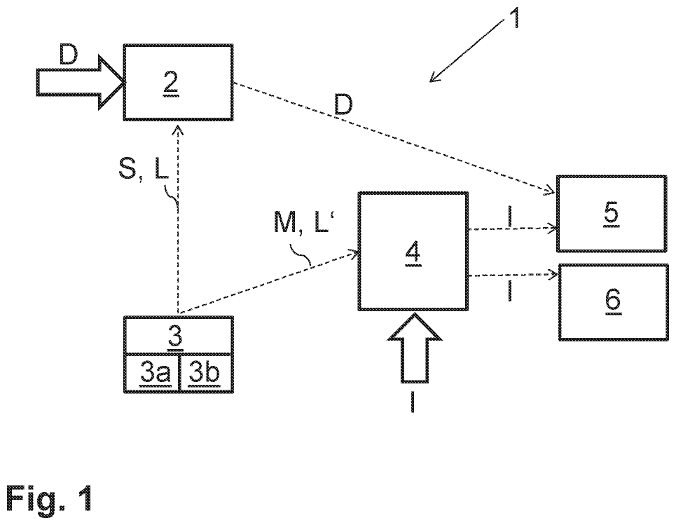

FIG. 1 shows a schematic representation of a system/method according to the present invention;

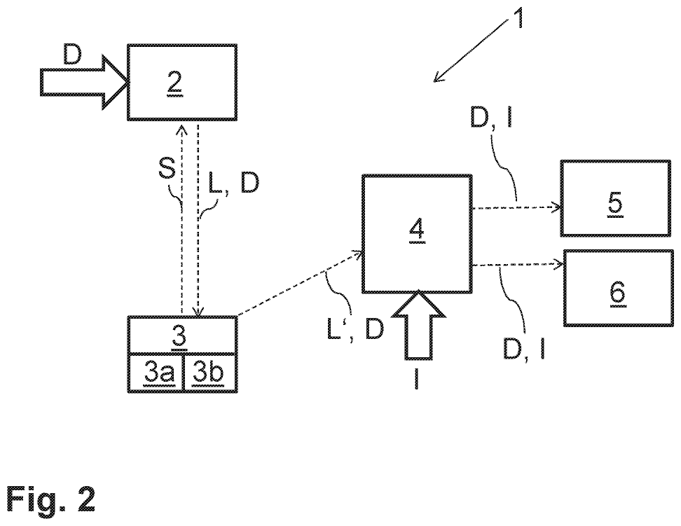

FIG. 2 shows a schematic representation of a further system/method according to the present invention;

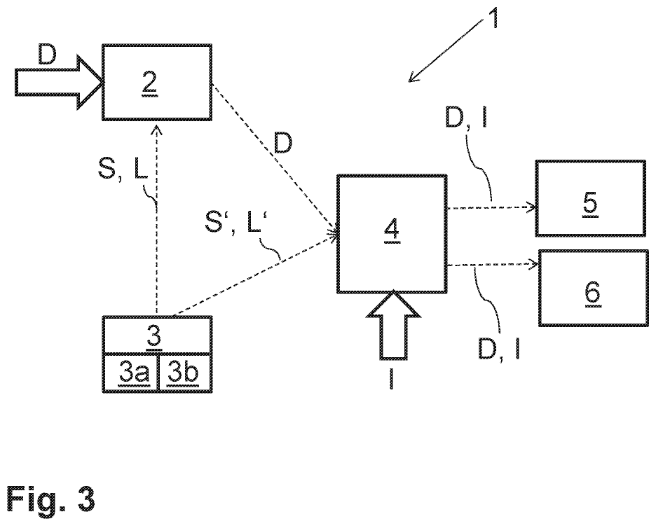

FIG. 3 shows a schematic representation of a further system/method according to the present invention;

The invention system 1 and method according to the present invention may be carried out in different embodiments/implementations.

One conceivable implementation shown in FIG. 1 relates to a system 1 that comprises a remote control device 3 having a button 3a (or some other form of user input) and a means of wireless communication 3b, e.g. a transmitter. When the patient takes an action (e.g., presses the device's button 3a), the remote control device 3 sends a control signal S via a wireless link L to a medical device 2 of the system 1, which medical device 2 is implanted into the patient, indicating that the medical device 2 should activate a recording of physiological data D (e.g. an ECG) of the patient. Particularly, in all embodiments described herein, the medical device 2 can be e.g. a cardiac pacemaker or a loop recorder or another medical device. In parallel or subsequently, the remote control device 3 sends a message M via a wireless link L′ to a patient device 4 (e.g., a message to a smartphone via Bluetooth, Bluetooth LE, NFC, etc.) notifying it that a recording of said data D by the medical device 2 has been requested. The patient device 4 may then display a notification to the patient. When the patient acknowledges the notification, the patient device 4 brings up a patient health survey that allows the patient to enter a health status I of the patient when the event happened. The patient may enter the information I in a variety of ways (e.g., button pushes, text, speech, photos and/or video). When done, the patient may send the health survey data I to the clinician 6 via a variety of mechanisms (e.g. email, fax, etc.). The patient device 4 may also send the information I automatically to a data storage facility 5 such as secure cloud or data service center. Alternatively, the health survey data I may be stored on the patient device 4 for later review at the clinician's 6 office. The medical device 2 keeps a record of the recorded event for later review by the clinician and/or may also send its recorded information D to the data storage facility 5. The information from the medical device 2 and the external patient device 4 may be correlated and presented to the clinician 6 as a cohesive data set.

In a second implementation/embodiment of the present invention as shown in FIG. 2, the medical device 2 may initiate recording of the physiological data D as described above in conjunction with FIG. 1. However, in contrast to FIG. 1, the medical device 2 now sends the recorded physiological data D to the remote control device 3 via a wireless link L. This remote control device 3 then passes the recorded data D to the patient device 4 via a wireless link L′. The patient device 4 then prompts the patient to input said information I concerning the patient's health status at the time of the event that lead the patient to initiate the recording. Further, the patient device 4 may extract some or all information (e.g. recorded physiological data D) from the medical device 2 to display it to the patient, and send the recorded data D from the medical device 2 combined/in addition with the health survey data I given by the patient to the clinician and/or data storage facility (e.g. cloud or data service center) 5.

In a third implementation/embodiment of the present invention as shown in FIG. 3 the remote control device 3 triggers a recording of said physiological data of the patient by the medical device 2 via sending a control signal S over a wireless link L, and further triggers the patient device 4 to start listening for a data transmission from the medical device 2 by sending a further control signal S′ to the patient device 4 via a wireless link L′. The medical device 2 then wirelessly transmits the recorded physiological data D directly to the patient device 4. The patient device 4 receives said data D from the medical device 2, and prompts the patient to provide said health survey information I and sends all information I, D to the clinician 6, and/or to a data storage facility 5 (such as a secure cloud or data service center). In addition, the patient device 4 may extract some or all information D from the medical device 2 to display it to the patient.

According to an embodiment of the present invention, the medical device 2 is an implantable medical device.

1. A system for collecting information concerning a patient's health status at an event, comprising:
a medical device configured for recording of physiological data (D) of a patient,
a remote control device for allowing the patient to initiate said recording of physiological data at an event, wherein the remote control device is configured to send a control signal to the medical device in response to a corresponding operation of the remote control device by the patient for initiating a recording of physiological data by the medical device, wherein the medical device is configured to record said physiological data when receiving said control signal, and
a patient device configured to prompt the patient after initiation of said recording of physiological data to input information on the patient's health status at the time of said event that lead the patient to initiate said recording of physiological data.

2. The system according to claim 1, wherein said physiological data is an electrocardiogram or a pressure signal.

3. The system according to claim 1, wherein the patient device is configured to record said information on the patient's health status in form of text, an audio signal, photos, video or any combination thereof.

4. The system according to claim 1, wherein the remote control device and the patient device are formed by a single device.

5. The system according to claim 1, wherein the patient device is configured to at least one of: store recorded physiological data, store said information on the patient's health status, and send recorded physiological data and/or said information on the patient's health status to a clinician and/or a data storage facility.

6. The system according to claim 1, wherein the patient device is configured to provide said information on the patient's health status with an identifier, and wherein the medical device is configured to provide the recorded physiological data with a correlated identifier.

7. The system according to claim 1, wherein the remote control device comprises a control element, and a transmitter, wherein the remote control device is configured to send said control signal via a wireless link using said transmitter when the patient actuates said control element to the medical device.

8. The system according to claim 1, wherein the remote control device is configured to send a message via a wireless link to the patient device in order to notify it that a recording of physiological data of the patient has been initiated, wherein the patient device is configured to display a notification to the patient and to prompt the patient to acknowledge said notification, and wherein when the patient acknowledges said notification, the patient device is configured to prompt the patient to input said information on the patient's health status.

9. The system according to claim 1, wherein the medical device is configured to send the recorded physiological data to the remote control device via a wireless link, wherein the remote control device is configured to send the recorded physiological data to the patient device via a wireless link.

10. The system according to claim 1, wherein the remote control device is configured to initiate said recording by sending said control signal via a wireless link to the medical device, wherein the remote control device is further configured to trigger the patient device to start listening for a transmission of the recorded physiological data from the medical device by sending a further control signal via a wireless link to the patient device, and wherein the medical device is configured to wirelessly transmit said recorded physiological data directly to the patient device.

11. A method for recording physiological data and information on a patient's health status, comprising the steps of:
initiating recording of physiological data of a patient by a medical device, wherein said recording is initiated by sending a control signal by a remote control device to the medical device,
receiving the recorded physiological data with a patient device or a data storage facility,
after initiation of said recording of physiological data prompting the patient to input information on the patient's health status at the time of an event that lead the patient to initiate said recording of physiological data.
